(19) United States
(12) Patent Application Publication
(10) Pub. No.: US 2013/0184703 A1
(43) Pub. Date: Jul. 18, 2013

(54) RENAL NERVE MODULATION DEVICES AND METHODS FOR MAKING AND USING THE SAME

(71) Applicant: BOSTON SCIENTIFIC SCIMED, INC., Maple Grove, MN (US)

(72) Inventors: BRICE L. SHIREMAN, MAPLE GROVE, MN (US); BRIAN R. REYNOLDS, RAMSEY, MN (US); KATHERINE ROUTH, COON RAPIDS, MN (US); BRIAN J. HANSON, SHOREVIEW, MN (US)

(73) Assignee: BOSTON SCIENTIFIC SCIMED, INC., MAPLE GROVE, MN (US)

(21) Appl. No.: 13/631,315

(22) Filed: Sep. 28, 2012

Related U.S. Application Data
(60) Provisional application No. 61/587,636, filed on Jan. 17, 2012.

(51) Int. Cl. A61B 18/14

(52) U.S. Cl. 606/41

(57) ABSTRACT

Medical devices and methods for making and using medical devices are disclosed. An example medical device may include a renal nerve modulation device. The renal nerve modulation device may include an elongate catheter shaft having a distal portion. An ablation member may be coupled to the distal portion. The catheter shaft may have a slotted portion having a plurality of slots formed therein. At least some of the slots formed in the slotted portion may define a plurality of beams in the slotted portion that extend along the slotted portion and that are aligned in a wave pattern.

### CROSS-REFERENCE TO RELATED APPLICATIONS

This application claims priority under 35 U.S.C. §119 to U.S. Provisional Application Ser. No. 61/587,636, filed Jan. 17, 2012, the entirety of which is incorporated herein by reference.

### TECHNICAL FIELD

The present disclosure pertains to medical devices, and methods for manufacturing medical devices. More particularly, the present disclosure pertains to deflectable medical devices and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device may include a renal nerve modulation device. The renal nerve modulation device may include an elongate catheter shaft having a distal portion. An ablation member may be coupled to the distal portion. The catheter shaft may have a slotted portion having a plurality of slots formed therein. At least some of the slots formed in the slotted portion may define a plurality of beams in the slotted portion that extend along the slotted portion and that are aligned in a wave pattern.

Another example renal nerve modulation device may include an elongate catheter shaft having a distal portion. An ablation member may be coupled to the distal portion. The catheter shaft may have a distal slotted portion having a plurality of slots formed therein and a proximal slotted portion having a plurality of slots formed therein. At least some of the slots formed in the distal slotted portion may define a plurality of longitudinally-aligned beams in the distal slotted portion. At least some of the slots formed in the proximal slotted portion may define a plurality of beams in the proximal slotted portion that extend along the proximal slotted portion and that are aligned in a wave pattern.

Also disclosed are methods including methods for treating hypertension. An example method may include providing a renal nerve modulation device. The renal nerve modulation device may include an elongate catheter shaft having a distal portion. An ablation member may be coupled to the distal portion. The catheter shaft may have a slotted portion having a plurality of slots formed therein. At least some of the slots formed in the slotted portion may define a plurality of beams in the slotted portion that extend along the slotted portion and that are aligned in a wave pattern. The method may also include advancing the renal nerve modulation device through a blood vessel to a position within the renal artery and activating the ablation member.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view illustrating an example renal nerve modulation system;

FIG. 2 is a schematic view illustrating the location of the renal nerves relative to the renal artery;

FIG. 3 is a longitudinally cut and flattened view of a portion of an example catheter shaft;

FIG. 4 is a side view of a portion of an example catheter;

FIG. 4A is a side view of a portion of another example catheter;

FIG. 5 is a partial cross-sectional side view of the example catheter illustrated in

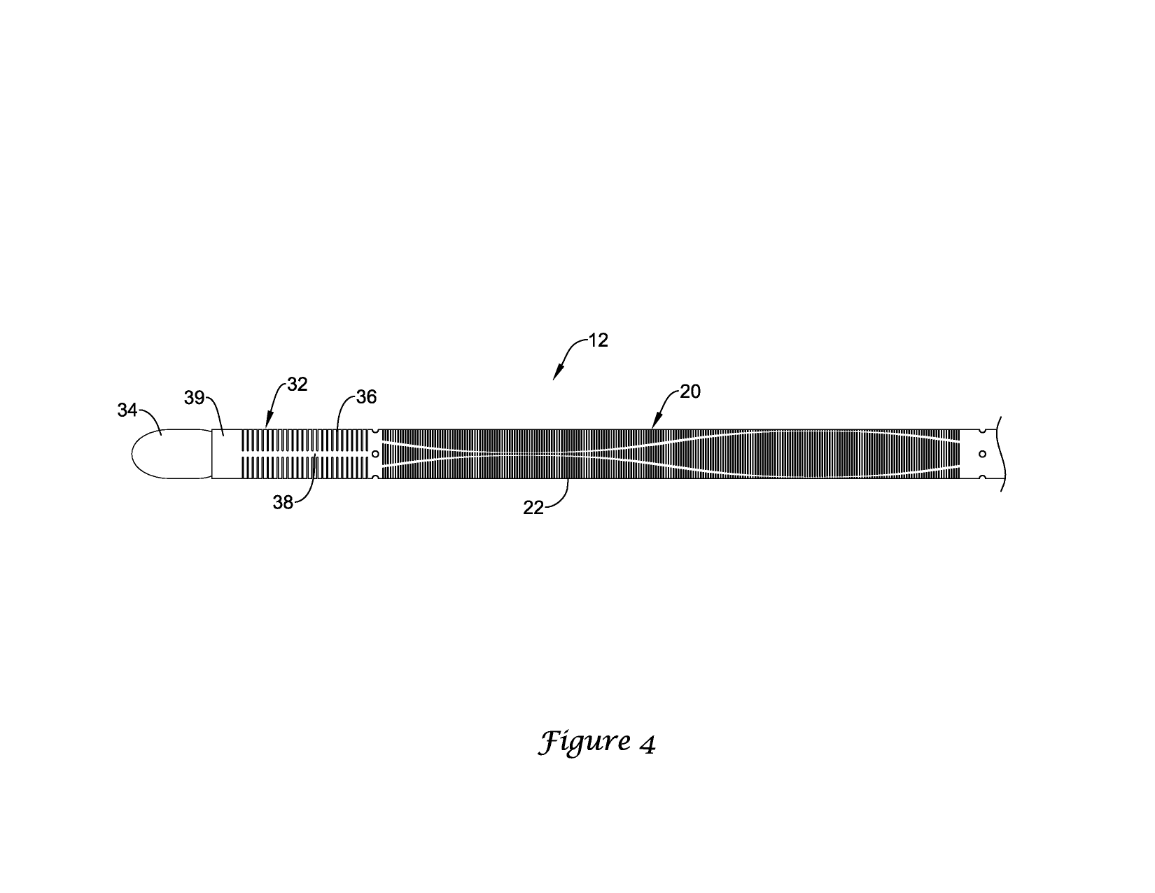

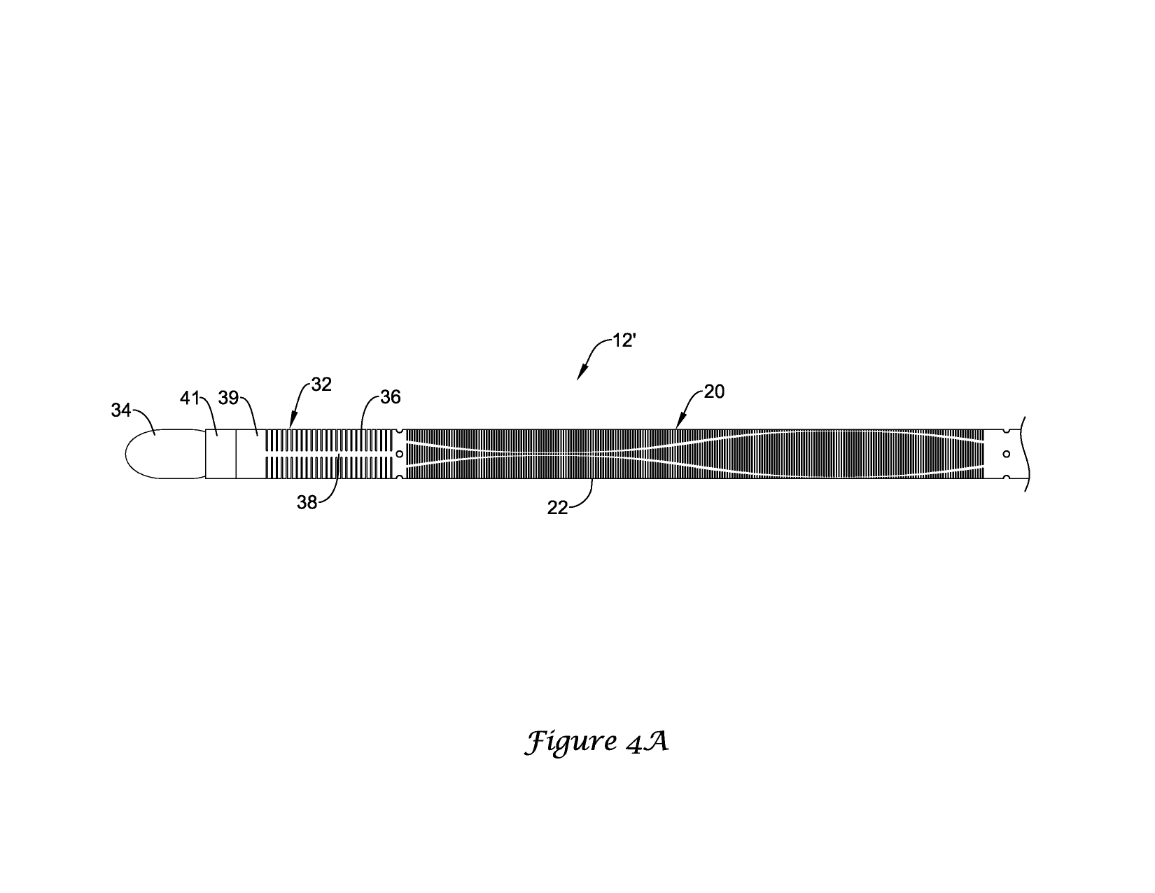

FIG. 4 disposed within a body lumen;

FIG. 6 is a perspective view of a portion of another example catheter shaft;

FIG. 7 is a perspective view of a portion of another example catheter shaft; and

FIG. 8 is a partial cross-sectional side view of the example catheter illustrated in

FIG. 7 disposed within a body lumen.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless clearly stated to the contrary.

Certain treatments may require the temporary or permanent interruption or modification of select nerve function. One example treatment is renal nerve ablation which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

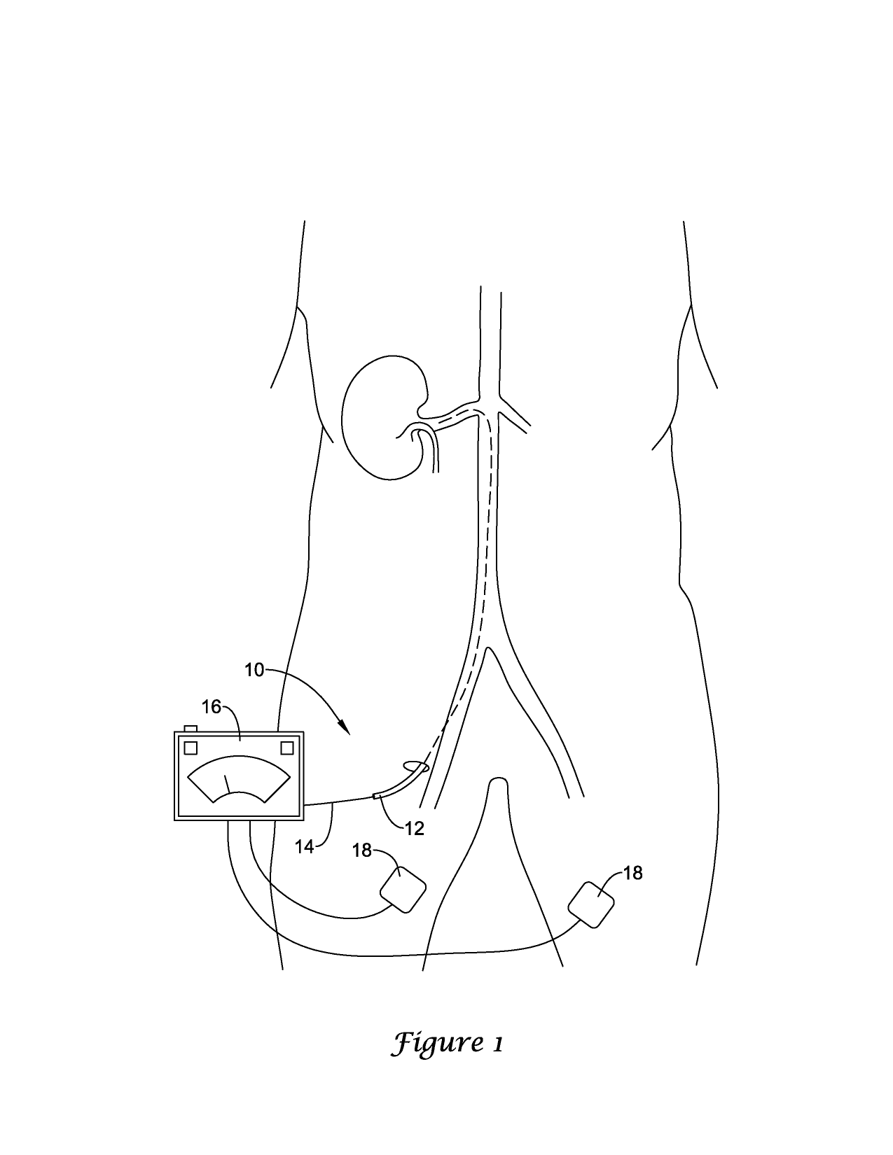

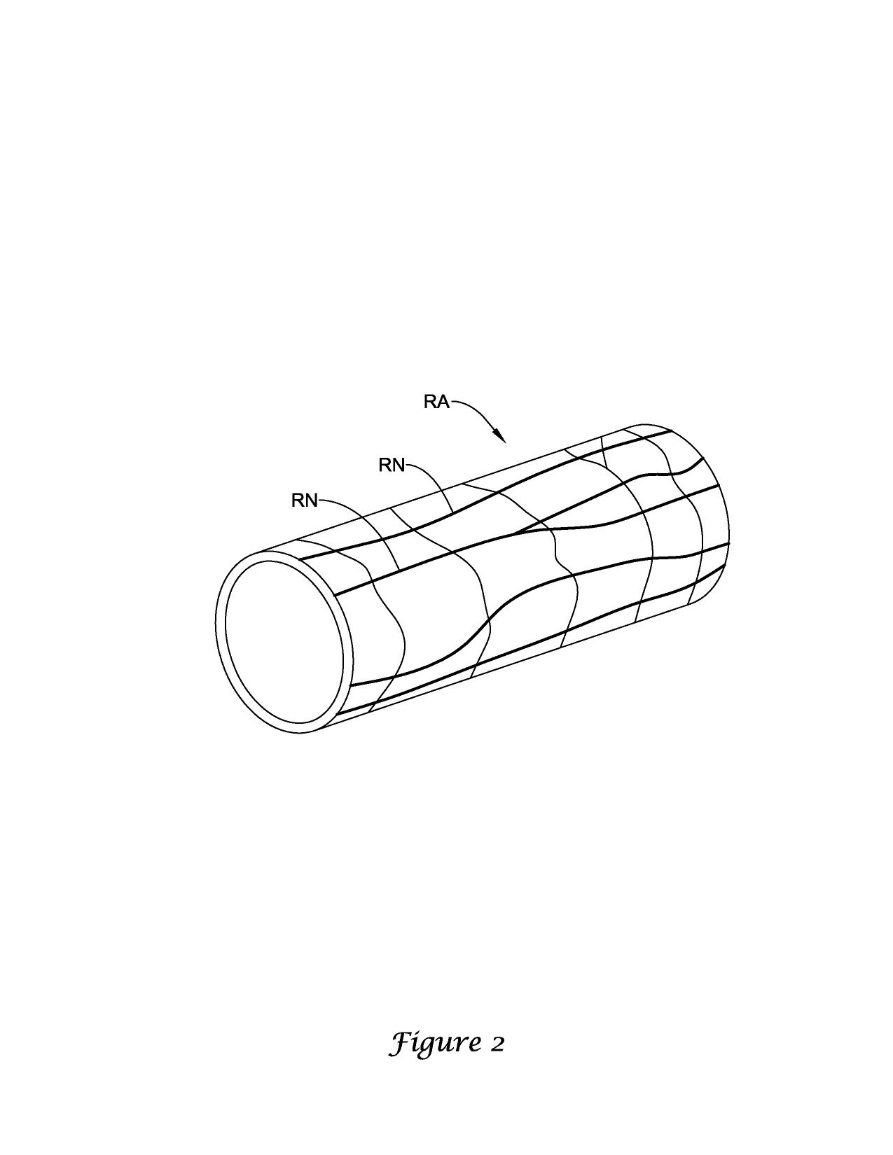

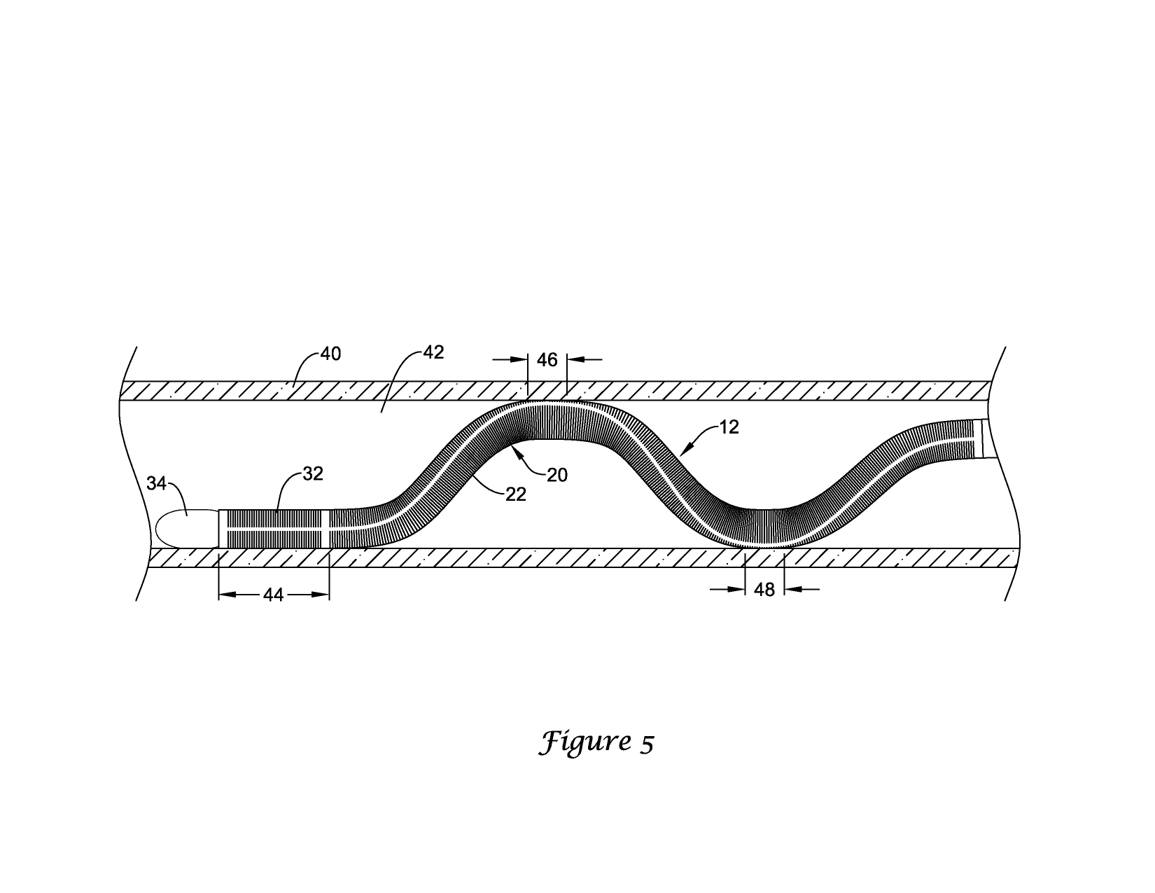

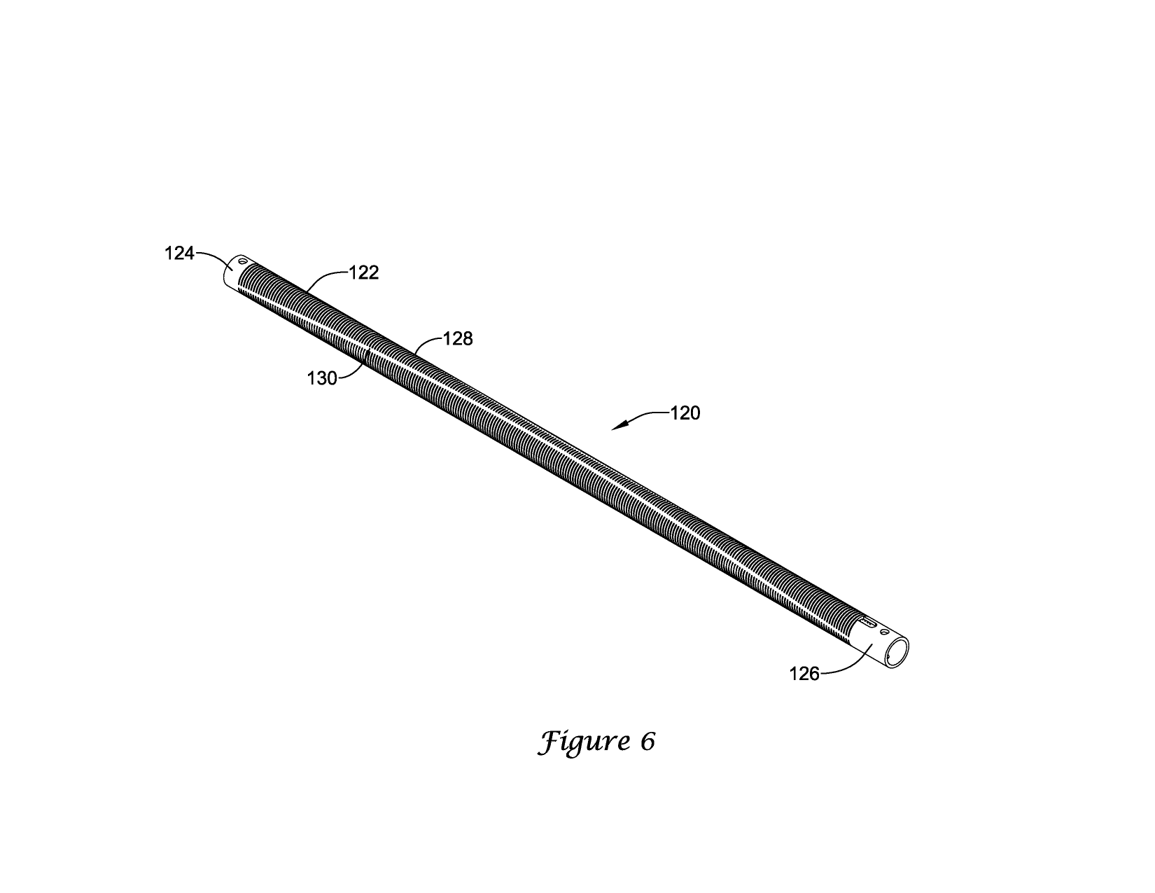

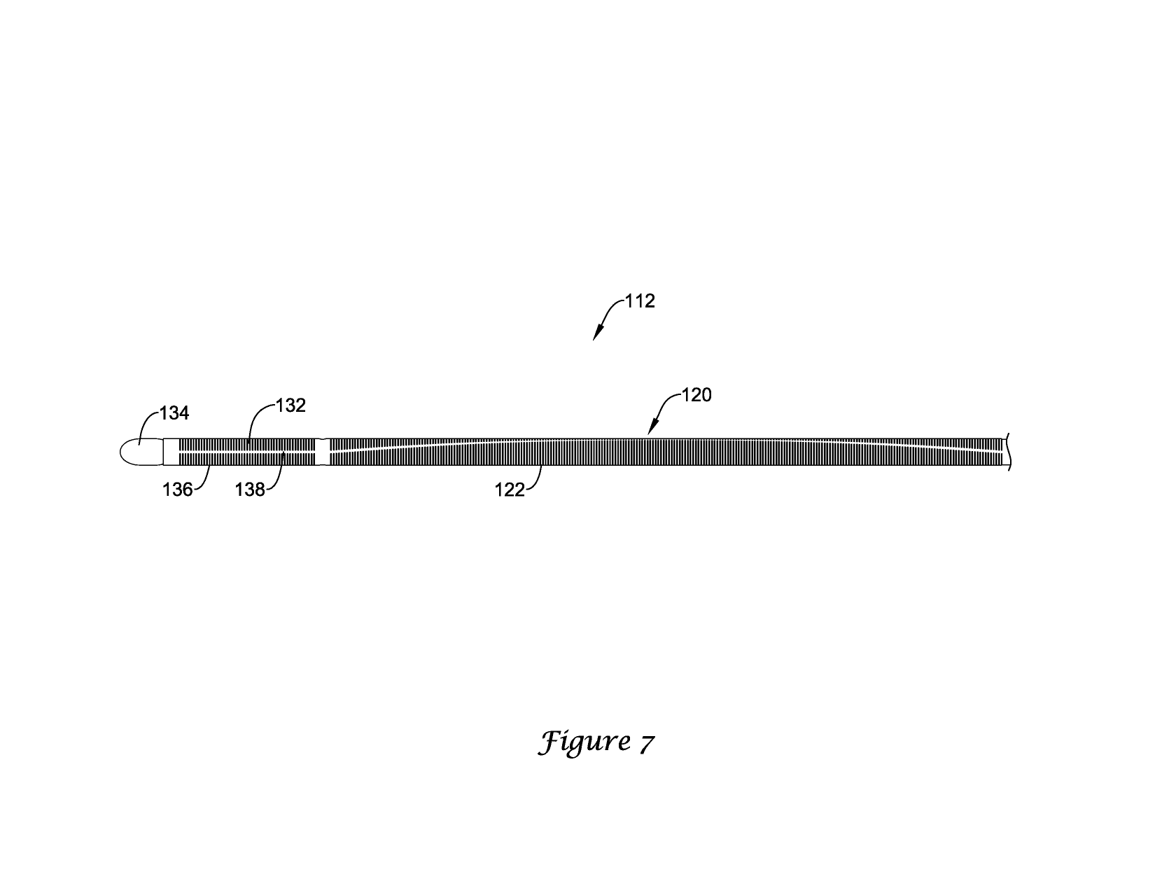

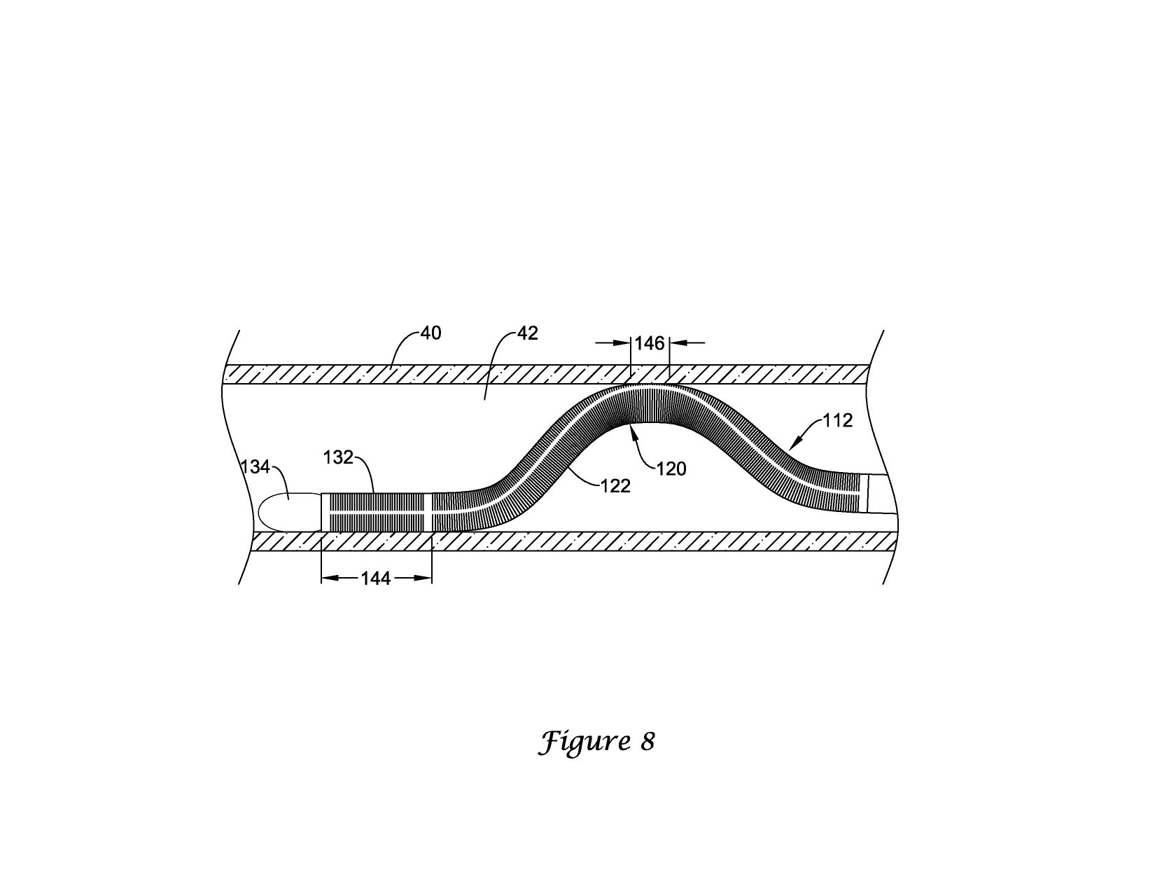

FIG. 1 is a schematic view of an example renal nerve modulation system 10 in situ. System 10 may include a renal ablation catheter 12 and one or more conductive element(s) 14 for providing power to catheter 12. A proximal end of conductive element(s) 14 may be connected to a control and power element 16, which supplies necessary electrical energy to activate one or more electrodes (e.g., ablation member or electrode 34 as shown in FIGS. 4-5) disposed at or near a distal end of catheter 12. When suitably activated, the electrodes are capable of ablating adjacent tissue. The terms electrode and electrodes may be considered to be equivalent to elements capable of ablating adjacent tissue in the disclosure which follows. In some instances, return electrode patches 18 may be supplied on the legs or at another conventional location on the patient's body to complete the circuit.

Control and power element 16 may include monitoring elements to monitor parameters such as power, temperature, voltage, amperage, impedance, pulse size and/or shape and other suitable parameters, with sensors mounted along catheter, as well as suitable controls for performing the desired procedure. In some embodiments, power element 16 may control a radio frequency (RF) electrode. The electrode may be configured to operate at a frequency of approximately 460 kHz. It is contemplated that any desired frequency in the RF range may be used, for example, from 450-500 kHz. It is further contemplated that additionally and/or other ablation devices may be used as desired, for example, but not limited to resistance heating, ultrasound, microwave, and laser devices and these devices may require that power be supplied by the power element 16 in a different form.

FIG. 2 illustrates a portion of the renal anatomy in greater detail. More specifically, the renal anatomy includes renal nerves RN extending longitudinally along the lengthwise dimension of renal artery RA and generally within or near the adventitia of the artery. The human renal artery wall is typically about 1 mm thick of which 0.5 mm is the adventitial layer. As will be seen in the figure, the circumferential location of the nerves at any particular axial location may not be readily predicted. Nerves may be difficult to visualize in situ and so treatment methods may desirably rely upon ablating multiple sites to ensure nerve modulation.

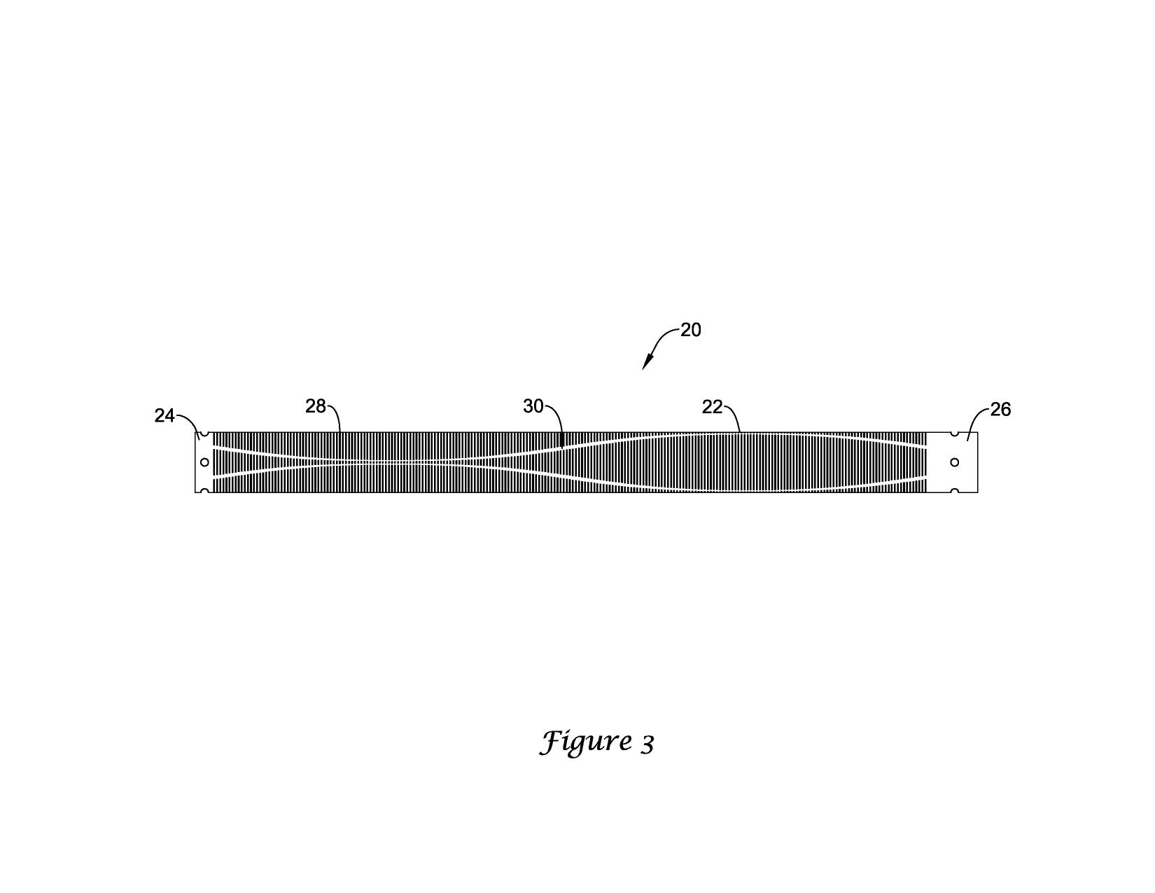

In order to efficiently ablate target nerves adjacent to the renal artery, it may be desirable for catheter 12 to be flexible and/or otherwise configured so that ablation member 34 may be positioned appropriately within the renal artery. This may include the use of catheters and/or catheter shaft sections that have desirable bending characteristics. FIG. 3 is a longitudinally cut and flattened view of a portion of a catheter shaft 20. Catheter shaft 20 may include a tubular member 22 having a distal end or region 24 and a proximal end or region 26. Tubular member 22 may have a plurality of slots 28 formed therein. The slots 28 may be arranged so as to define a plurality of beams 30 (e.g., portions of tubular member 22 that remain after forming slots 28 therein).

Beams 30 may be arranged in a number of different manners so as to define a pattern. In at least some embodiments, the pattern of beams 30 may be a wave or wave-like pattern. For example, the pattern of beams 30 may be a sine wave pattern as shown in FIG. 3. The sine-wave pattern may be derived from the general equation: y=A*sin(B*x)+C. Other patterns are contemplated including a half-sine wave pattern, a cosine wave pattern (e.g., derived from the general equation: y=A*cos(B*x)+C), a half-cosine wave pattern, other patterns based on trigonometric functions (e.g., tangent, secant, cosecant, cotangent, and/or combinations thereof), other wave patterns, non-wave or non-repetitive patterns, patterns based on mathematical functions (including exponential, polynomial, power, combinations thereof, or the like), or the like. For the purposes of this disclosure, a half-sine and half-cosine wave pattern may be understood to be a wave pattern of oscillations where only the portions of the sine/cosine wave having a positive amplitude are utilized. In other words, if a sine or cosine wave can be understood as having both peaks and valleys, a half-sine or half-cosine wave may be understood to have only the peaks. In addition to these patterns, other patterns may also be utilized and a variety of these patterns are contemplated. For examples, other oscillating patterns, squared patterns, random patterns, or other patterns may be utilized. The pattern of beams 30 may be defined by longitudinally-aligned beams extending along tubular member 22 where adjacent beams are (in addition to being longitudinally spaced) spatially and/or radially shifted relative to one another around tubular member 22 to form the pattern. Alternatively, a plurality of beams or a group (e.g., a “first” group) of longitudinally-adjacent beams may be longitudinally-aligned with one another and subsequent beams and/or groups of beams may be spatially and/or radially shifted relative to the first group of beams around tubular member 22 to form the pattern.

The pattern of beams 30 may desirably impact the bending characteristics of catheter shaft 20 (and/or catheter 12). In at least some embodiments, the pattern of beams may be designed to bias catheter 12 to bend toward a certain direction when actuated (e.g., actuated actively using a pull wire or other suitable deflection mechanism) or otherwise encountering an obstacle. This may include a pattern that defines a “preferred bending direction” or “single-sided deflection” configuration for catheter 12. In addition, the pattern of beams 30 may define one or more discrete bending regions or bending points where bending in a desired direction occurs. For example, the pattern of beams 30 may define one, two, or more discrete bending points where catheter shaft 20 is configured to bend.

Moreover, slotted tubular members like tubular member 22 may be designed to bend with a relatively low actuation force, may be tailored to a particular bending pattern, and/or be formed with a robust or simple cut pattern. This may include bending with or without an active actuation mechanism. Collectively, these design considerations may allow catheter 12 to be suited for using as a part of intervention where fine and/or tunable bending may aid the intervention. This may include renal nerve modulation (e.g., as part of a treatment for hypertension), placement of cardiac leads, other cardiac interventions, neurological interventions, gastrological interventions, or the like.

FIG. 4 illustrates a portion of catheter 12 including catheter shaft 20 and an ablation member or electrode 34 coupled to catheter shaft 20. Ablation member 34 may be formed at or otherwise form a distal tip of catheter shaft 20. In general, ablation member 34 may be configured to ablate target tissue at or near a body lumen. For example, ablation member 34 may be used to ablate a renal nerve adjacent to a renal artery. Ablation member 34 may vary and may include a number of structures such as a plurality of wires (e.g., two wires) that connect with conductive element 14 and, ultimately, control and power element 16. Ablation member 34 may also include other structures and/or features associated typically associated with ablation (e.g., thermal ablation) such as a temperature monitoring member, which may take the form of a thermocouple or thermistor. In at least some embodiments, a thermistor including two thermistor wires may be disposed adjacent to ablation member 34. In some embodiments, the wires are not physically connected to ablation member 34. The thermistor wires may terminate in the center bore of the ablation member 34 and may be potted with a thermally conducting epoxy in a plastic tube which is then glued to the bore of the ablation member 34. These are just examples.

In at least some embodiments, ablation member 34 may include a radio frequency (RF) electrode. In some of these and in other embodiments, ablation member 34 may include a thermal electrode, an ultrasound transducer, a laser electrode, a microwave electrode, combinations thereof, or the like.

As can also be seen in FIG. 4, catheter shaft 20 may include tubular member 22 (e.g., as described herein) and a distal tubular member or flex tube 32. Flex tube 32 may have a plurality of slots 36 formed therein. A plurality of beams 38 may also be defined in flex tube 32. In general, flex tube 32 is configured to be flexible so that the distal portion of catheter 12 (e.g., adjacent to ablation member 34) can bend upon encountering the wall of a body lumen. Accordingly, flex tube 32 can bend when/if ablation member 34 engages the wall of the body lumen so that ablation member 34 may atraumatically follow along the wall of the body lumen. It should be noted that catheters are contemplated that include more than one flex tube, flex tube(s) positioned at alternative locations, or that lack flex tube 32.

Ablation member 34 may be coupled to catheter shaft 20, for example at or adjacent to flex tube 32. For example, ablation member 34 may be attached to a distal region 39 of flex tube 32 as shown in FIG. 4. In some embodiments, distal region 39 takes the form of an uncut region of flex tube 32. In other embodiments, distal region 39 may include a non-metallic (e.g., polymeric) section that is coupled to or otherwise attached to flex tube 32. Alternatively, FIG. 4A illustrates example catheter 12′ (which may be similar in form and function to other catheters disclosed herein) that includes a distal tubular region 41 coupled to or otherwise attached to flex tube 32 (e.g., at or adjacent to distal region 39). Region 41 may take the form of a non-metallic (e.g., polymeric) tube or filler material and may help to electrically insulate ablation member 34 from flex tube 32 (and/or tubular member 22 in embodiments that lack flex tube 32). Ablation member 34 may also be coupled to catheter shaft 20 at other locations including at the distal end of catheter 12, adjacent to (but longitudinally spaced from) the distal end of catheter 12, along flex tube 32, between flex tube 32 and tubular member 22, along tubular member 22 (including locations where ablation member 34 would be disposed along a curved portion of catheter 12, which may provide more force between ablation member 34 and the vessel wall and/or that may aid in providing a desirable positioning/orientation relative to the vessel wall), or at essentially any other suitable location.

Catheter 12 may also include an actuation mechanism (not shown) that may be used to actively bend or deflect catheter shaft 20. In at least some embodiments, the actuation mechanism may include a pull wire. The pull wire may be coupled to (e.g., with a weld, an adhesive, etc.) a distal portion of catheter shaft 20 (e.g., at or adjacent to ablation member 34, at or adjacent to flex tube 32, at or adjacent to a distal portion of tubular member 22, or the like). The pull wire may extend along the exterior of catheter shaft 20, along an interior region of catheter shaft 20, or both to a position where it may be accessible to a clinician and can be manipulated in order to deflect catheter shaft 20. The actuation mechanism may be utilized to deflect or otherwise bend catheter shaft 20.

FIG. 5 illustrates catheter 12 disposed within the lumen 42 of a blood vessel 40. Here it can be seen that flex tube 32 may allow a portion of catheter shaft 20 (e.g., along or adjacent to flex tube 32) to lay flat along the vessel wall and define a contact region 44. The sine-wave pattern of beams 30 in tubular member 22 may form or otherwise define a plurality of contact points or regions where catheter shaft 20 contacts the wall of vessel 40. For example, the sine-wave pattern of beams 30 may define a first contact region 46 and a second contact region 48 where catheter shaft 20 contacts the wall of vessel 40 (e.g., when actively actuated using a pull wire or other deflection mechanism). The arrangement of catheter 12 within vessel 40 with contact regions 46/48 may be described as an “S” configuration.

FIG. 6 illustrates a portion of catheter shaft 120, which may be similar to other catheter shafts disclosed herein. Catheter shaft 120 may include a tubular member 122 having distal end 124 and proximal end 126. Slots 128 may be formed in tubular member 122 and define a plurality of beams 130. According to this embodiment, beams 130 may be arranged in a half-sine wave pattern.

FIG. 7 illustrates catheter 112 including catheter shaft 120. According to this embodiment, catheter shaft 120 may include tubular member 122 and a distal tubular member or flex tube 132. Flex tube 132 may have a plurality of slots 136 formed therein. A plurality of beams 138 may also be defined in flex tube 132. An ablation member 134 may be coupled to catheter shaft 120, for example at or adjacent to flex tube 132.

FIG. 8 illustrates catheter 112 disposed within the lumen 42 of a blood vessel 40. Here it can be seen that flex tube 132 may allow a portion of catheter shaft 120 (e.g., along or adjacent to flex tube 132) to lay flat along the vessel wall and define a contact region 144. The pattern of beams 130 in tubular member 122 may form or otherwise define a contact point or region where catheter shaft 120 contacts the wall of vessel 40. For example, the pattern of beams 130 in tubular member 122 may define a singular contact region 146 where catheter shaft 120 contacts the wall of vessel 40.

The materials that can be used for the various components of catheter 12 (and/or other catheters disclosed herein) and the various bodies and/or members disclosed herein may include those commonly associated with medical devices. For simplicity purposes, the following discussion makes reference to catheter shaft 20 and other components of catheter 12. However, this is not intended to limit the devices and methods described herein, as the discussion may be applied to other similar tubular members and/or components of tubular members or devices disclosed herein.

Catheter shaft 20 and/or other components of catheter 12 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of catheter shaft 20 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of catheter 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of catheter 12 to achieve the same result.

In some embodiments, a degree of Magnetic Resonance Imaging (MRI) compatibility is imparted into catheter 12. For example, catheter shaft 20 or portions thereof, may be made of a material that does not substantially distort the image and create substantial artifacts (i.e., gaps in the image). Certain ferromagnetic materials, for example, may not be suitable because they may create artifacts in an MRI image. Catheter shaft 20 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of catheter shaft 20 that may define a generally smooth outer surface for catheter 12. In other embodiments, however, such a sheath or covering may be absent from a portion of all of catheter 12, such that catheter shaft 20 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the catheter 12 (including, for example, the exterior surface of catheter shaft 20) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of catheter shaft 20 or other portions of catheter 12. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, saccharides, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

In addition to variations in materials, various embodiments of arrangements and configurations are also contemplated for slots 28 (and/or other slots disclosed herein) formed in tubular member 22 and for slots 36 formed in flex tube 32 in addition to what is described above or may be used in alternate embodiments. For simplicity purposes, the following discussion makes reference to slots 28. However, this discussion may also be applicable to any of the cuts or slots disclosed herein as well as any of the beams disclosed herein. For example, in some embodiments, at least some, if not all of slots 28 are disposed at the same or a similar angle with respect to the longitudinal axis of catheter shaft 20. As shown, slots 28 can be disposed at an angle that is perpendicular, or substantially perpendicular, and/or can be characterized as being disposed in a plane that is normal to the longitudinal axis of catheter shaft 20. However, in other embodiments, slots 28 can be disposed at an angle that is not perpendicular, and/or can be characterized as being disposed in a plane that is not normal to the longitudinal axis of catheter shaft 20. Additionally, a group of one or more slots 28 may be disposed at different angles relative to another group of one or more slots 28. The distribution and/or configuration of slots 28 can also include, to the extent applicable, any of those disclosed in U.S. Pat. Publication No. US 2004/0181174, the entire disclosure of which is herein incorporated by reference.

Slots 28 may be provided to enhance the flexibility of catheter shaft 20 while still allowing for suitable torque transmission characteristics. Slots 28 may be formed such that one or more rings and/or tube segments interconnected by one or more segments and/or beams 30 that are formed in catheter shaft 20, and such tube segments and beams 30 may include portions of catheter shaft 20 that remain after slots 28 are formed in the body of catheter shaft 20. Such an interconnected structure may act to maintain a relatively high degree of torsional stiffness, while maintaining a desired level of lateral flexibility. In some embodiments, some adjacent slots 28 can be formed such that they include portions that overlap with each other about the circumference of catheter shaft 20. In other embodiments, some adjacent slots 28 can be disposed such that they do not necessarily overlap with each other, but are disposed in a pattern that provides the desired degree of lateral flexibility.

Additionally, slots 28 can be arranged along the length of, or about the circumference of, catheter shaft 20 to achieve desired properties. For example, adjacent slots 28, or groups of slots 28, can be arranged in a symmetrical pattern, such as being disposed essentially equally on opposite sides about the circumference of catheter shaft 20, or can be rotated by an angle relative to each other about the axis of catheter shaft 20. Additionally, adjacent slots 28, or groups of slots 28, may be equally spaced along the length of catheter shaft 20, or can be arranged in an increasing or decreasing density pattern, or can be arranged in a non-symmetric or irregular pattern. Other characteristics, such as slot size, slot shape, and/or slot angle with respect to the longitudinal axis of catheter shaft 20, can also be varied along the length of catheter shaft 20 in order to vary the flexibility or other properties. In other embodiments, moreover, it is contemplated that the portions of the tubular member, such as a proximal section, or a distal section, or the entire catheter shaft 20, may not include any such slots 28.

As suggested herein, slots 28 may be formed in groups of two, three, four, five, or more slots 28, which may be located at substantially the same location along the axis of catheter shaft 20. Alternatively, a single slot 28 may be disposed at some or all of these locations. Within the groups of slots 28, there may be included slots 28 that are equal in size (i.e., span the same circumferential distance around catheter shaft 20). In some of these as well as other embodiments, at least some slots 28 in a group are unequal in size (i.e., span a different circumferential distance around catheter shaft 20). Longitudinally adjacent groups of slots 28 may have the same or different configurations. For example, some embodiments of catheter shaft 20 include slots 28 that are equal in size in a first group and then unequally sized in an adjacent group. It can be appreciated that in groups that have two slots 28 that are equal in size and are symmetrically disposed around the tube circumference, the centroid of the pair of beams (i.e., the portion of catheter shaft 20 remaining after slots 28 are formed therein) is coincident with the central axis of catheter shaft 20. Conversely, in groups that have two slots 28 that are unequal in size and whose centroids are directly opposed on the tube circumference, the centroid of the pair of beams can be offset from the central axis of catheter shaft 20. Some embodiments of catheter shaft 20 include only slot groups with centroids that are coincident with the central axis of the catheter shaft 20, only slot groups with centroids that are offset from the central axis of catheter shaft 20, or slot groups with centroids that are coincident with the central axis of catheter shaft 20 in a first group and offset from the central axis of catheter shaft 20 in another group. The amount of offset may vary depending on the depth (or length) of slots 28 and can include other suitable distances.

Slots 28 can be formed by methods such as micro-machining, saw-cutting (e.g., using a diamond grit embedded semiconductor dicing blade), electrical discharge machining, grinding, milling, casting, molding, chemically etching or treating, or other known methods, and the like. In some such embodiments, the structure of the catheter shaft 20 is formed by cutting and/or removing portions of the tube to form slots 28. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing catheter 12 may include forming slots 28 in catheter shaft 20 using these or other manufacturing steps.

In at least some embodiments, slots 28 may be formed in tubular member using a laser cutting process. The laser cutting process may include a suitable laser and/or laser cutting apparatus. For example, the laser cutting process may utilize a fiber laser.

Utilizing processes like laser cutting may be desirable for a number of reasons. For example, laser cutting processes may allow catheter shaft 20 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form catheter shaft 20 without being limited by a minimum cutting blade size. Consequently, tubular members may be fabricated for use in neurological devices or other devices where a relatively small size may be desired.

### EXAMPLES

The disclosure may be further clarified by reference to the following Examples, which serve to exemplify some of the preferred embodiments, and not to limit the invention in any way.

### Example 1

An example tubular member was modeled using SOLIDWORKS software (commercially available from Dassault Systèmes SolidWorks Corp. Waltham, Mass., USA). The tubular member, which may be used as a model of tubular member 22, was designed to have a plurality of slots. The slots were defined by opposed pairs of cuts formed in the tubular member. Beams were defined between the opposed cuts. The beams were arranged in a sine-wave pattern along the tubular member. The sine-wave pattern was derived from the general equation:

y=A*sin(B*x)+C Equation (1)

In this equation, y is the beam length and A, B, and C are constants. In one embodiment, the modeled tubular member was a 0.936 tubular member and the constants were derived from the boundary conditions solved at x=0, x=0.234, and x=0.468. To generate the sine-wave pattern, the following equations were inputted into SOLIDWORKS to generate the sine-wave beam pattern:

y=(0.018−0.0197*π)sin(π*x/0.468)+(0.197*π+0.006) Equation (2)

y=(0.0197*π−0.018)sin(π*x/0.468)+(0.197*π+0.006) Equation (3)

### Example 2

An example nickel-titanium alloy (e.g., nitinol) tubular member having an inner diameter of 0.032 inches and an outer diameter of 0.0395 inches was cut using a laser cutting process to have a sine-wave pattern of beams using the model described in Example 1.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A renal nerve modulation device, comprising:
an elongate catheter shaft having a distal portion;
an ablation member coupled to the distal portion;
wherein the catheter shaft has a slotted portion having a plurality of slots formed therein; and
wherein at least some of the slots formed in the slotted portion define a plurality of beams in the slotted portion that extend along the slotted portion and that are aligned in a pattern.

2. The renal nerve modulation device of claim 1, wherein the plurality of beams that extend along the slotted portion are aligned in a sine wave pattern.

3. The renal nerve modulation device of claim 1, wherein the plurality of beams that extend along the slotted portion are aligned in a half sine wave pattern.

4. The renal nerve modulation device of claim 1, wherein the plurality of beams that extend along the slotted portion are aligned in a cosine wave pattern.

5. The renal nerve modulation device of claim 1, wherein the plurality of beams that extend along the slotted portion are aligned in a half cosine wave pattern.

6. The renal nerve modulation device of claim 1, wherein the slotted portion is configured to define a single point of contact with a vessel wall when disposed within a blood vessel.

7. The renal nerve modulation device of claim 1, wherein the slotted portion is configured to define two points of contact with a vessel wall when disposed within a blood vessel.

8. The renal nerve modulation device of claim 1, further comprising a flex tube disposed at a distal end of the slotted portion, the flex tube has a plurality of slots formed therein.

9. The renal nerve modulation device of claim 8, wherein at least some of the slots formed in the flex tube define a plurality of beams in the flex tube that are longitudinally-aligned.

10. The renal nerve modulation device of claim 1, wherein the catheter shaft is configured to have a preferred bending direction.

11. The renal nerve modulation device of claim 1, wherein the ablation member includes a radio frequency electrode.

12. The renal nerve modulation device of claim 1, wherein the plurality of beams in the slotted portion are aligned in a wave pattern.

13. A renal nerve modulation device, comprising:
an elongate catheter shaft having a distal portion;
an ablation member coupled to the distal portion;
wherein the catheter shaft has a distal slotted portion having a plurality of slots formed therein and a proximal slotted portion having a plurality of slots formed therein;
wherein at least some of the slots formed in the distal slotted portion define a plurality of longitudinally-aligned beams in the distal slotted portion; and
wherein at least some of the slots formed in the proximal slotted portion define a plurality of beams in the proximal slotted portion that extend along the proximal slotted portion and that are aligned in a wave pattern.

14. The renal nerve modulation device of claim 13, wherein the plurality of beams that extend along the proximal slotted portion are aligned in a sine wave pattern.

15. The renal nerve modulation device of claim 14, wherein the proximal slotted portion is configured to define two points of contact with a vessel wall when disposed within a blood vessel.

16. The renal nerve modulation device of claim 13, wherein the plurality of beams that extend along the proximal slotted portion are aligned in a half sine wave pattern.

17. The renal nerve modulation device of claim 16, wherein the proximal slotted portion is configured to define a single point of contact with a vessel wall when disposed within a blood vessel.

18. The renal nerve modulation device of claim 13, wherein the ablation member includes a radio frequency electrode.

19. A method for treating hypertension, the method comprising:
providing a renal nerve modulation device, the renal nerve modulation device comprising:
an elongate catheter shaft having a distal portion,
an ablation member coupled to the distal portion,
wherein the catheter shaft has a slotted portion having a plurality of slots formed therein, and
wherein at least some of the slots formed in the slotted portion define a plurality of beams in the slotted portion that extend along the slotted portion and that are aligned in a wave pattern;
advancing the renal nerve modulation device through a blood vessel to a position within a renal artery; and
activating the ablation member.

20. The method of claim 19, wherein activating the ablation member includes placing the ablation member adjacent to a wall of the renal artery.
